(19) United States
(12) Patent Application Publication
(10) Pub. No.: US 2005/0256417 A1
(43) Pub. Date: Nov. 17, 2005

(54) Emergency room triage system

(76) Inventors: David R. Fischell, Fair Haven, NJ (US); Jonathan Harwood, Rumson, NJ (US)

Correspondence Address: ROSENBERG, KLEIN & LEE, 3458 ELLICOTT CENTER DRIVE-SUITE 101, ELLICOTT CITY, MD, 21043, US

(21) Appl. No.: 10/844,411

(22) Filed: May 13, 2004

(51) Int. Cl. A61N 1/362; A61B 5/0402

(52) U.S. Cl. 600/510; 607/4

(57) ABSTRACT

A patient diagnostic emergency room triage system includes an implanted cardiac device which may be a cardiosaver, pacemaker or cardiac defibrillator for recording electrogram data associated with the detection of a cardiac event. A communication mechanism receives electrogram data from the implanted cardiac device and a visual display displays the electrogram data recorded by the implanted cardiac device. The visual display permits displaying electrogram data associated with an ST segment related cardiac event.

### FIELD OF USE

This invention is in the field of systems, including devices with diagnostic capabilities implanted within a human patient.

### BACKGROUND OF THE INVENTION

Heart disease is the leading cause of death in the United States. A heart attack (also known as an acute myocardial infarction (AMI)) typically results from a thrombus (i.e., a blood clot) that obstructs blood flow in one or more coronary arteries. AMI is a common and life-threatening complication of coronary heart disease. Myocardial ischemia is caused by an insufficiency of oxygen to the heart muscle. Ischemia is typically provoked by physical activity or other causes of increased heart rate when one or more of the coronary arteries are narrowed by atherosclerosis. Patients will often (but not always) experience chest discomfort (angina) when the heart muscle is experiencing ischemia. Those with coronary atherosclerosis are at higher risk for AMI if the plaque becomes further obstructed by thrombus.

The two most significant problems faced in treating AMI are:
1. the time delay from the onset of symptoms until arrival at a medical care facility. Currently in the United States this time delay is approximately 3 hours, and
2. the additional time (often an hour or more) that it takes once the patient arrives at the medical care facility or emergency room until AMI is diagnosed and a therapy is provided.

Acute myocardial infarction and ischemia may be detected from a patient's electrocardiogram (ECG) by noting an ST segment voltage change and are therefore classified as ST segment related cardiac events. However, without knowing the patient's normal ECG pattern, detection from a standard 12-lead ECG can be unreliable. What is more, there is a significant time required to access a portable ECG machine, attach the leads to the patient, collect the ECG and then read and analyze the paper trace.

Fischell et al. in U.S. Pat. Nos. 6,112,116, 6,272,379 and 6,609,023 describe implantable systems and algorithms for detecting the onset of acute myocardial infarction and providing both treatment and alarming to the patient. These implantable systems include pacemakers, implantable cardiac defibrillators (ICDS) and purely diagnostic implants called cardiosavers. Fischell et al., in the above references, describes a physician's programmer as a laptop computer-like device designed to upload programming to the implant and download electrogram data collected by the implant. Also described is a hand-held computer designed to display alarm and baseline electrogram-related data. While these systems are designed to alert the patient to get him or her quickly to the emergency room, the Fischell et al. patents do not describe a means to quickly triage the patients in the emergency room to avoid the delays and inaccuracies currently found in the use of a 12-lead ECG to diagnose AMI.

Although often described as an electrocardiogram (ECG), the stored electrical signal from the heart as measured from electrodes within the body should be termed an “electrogram”. The early detection of an acute myocardial infarction or exercise-induced myocardial ischemia caused by an increased heart rate or exertion is feasible using a system that can detect a change in a patient's electrogram. The portion of such a system that includes the means to detect a cardiac event is defined herein as a “cardiosaver,” and the entire system including the cardiosaver and the external portions of the system is defined herein as a “guardian system.”

While pacemaker and ICD programmers can download and display electrogram data, they are generally large complex machines, are not easily attached to a wall in an emergency room, and are not designed to automatically download and display ST-segment-related cardiac event electrogram data. In addition pacemakers and ICDs currently use high pass filtering that is unsuitable for use in the detection of ST segment elevation or depression. What is more, they do require extensive training to access downloaded electrogram data.

Furthermore, although the masculine pronouns “he” and “his” are used herein, it should be understood that the patient or the medical practitioner who treats the patient could be a man or a woman. Still further the term; “medical practitioner” shall be used herein to mean any person who might be involved in the medical treatment of a patient. Such a medical practitioner would include, but is not limited to, a medical doctor (e.g., a general practice physician, an internist or a cardiologist), a medical technician, a paramedic, a nurse or an electrogram analyst. A “cardiac event” can be ST segment related event such as an acute myocardial infarction or ischemia caused by effort (such as exercise). A cardiac event can also be arrhythmia. Examples of arrhythmia cardiac events include an elevated heart rate, bradycardia, tachycardia, atrial fibrillation, atrial flutter, ventricular fibrillation, and premature ventricular or atrial contractions (PVCs or PACs respectively).

For the purpose of this invention, the term “electrocardiogram” is defined to be the heart's electrical signals sensed by means of skin surface electrodes that are placed in a position to indicate the heart's electrical activity (depolarization and repolarization). An electrocardiogram segment refers to the recording of electrocardiogram data for either a specific length of time, such as 10 seconds, or a specific number of heart beats, such as 10 beats. For the purposes of this specification, the PQ segment of a patient's electrocardiogram is the typically flat segment of a beat of an electrocardiogram that occurs just before the R wave. A beat is defined as a sub-segment of an electrocardiogram segment containing exactly one R wave.

For the purpose of this invention, the term “electrogram” is defined to be the heart's electrical signals from one or more implanted electrode(s) that are placed in a position to indicate the heart's electrical activity (depolarization and repolarization). An electrogram segment refers to the recording of electrogram data for either a specific length of time, such as 10 seconds, or a specific number of heart beats, such as 10 beats. For the purposes of this specification, the PQ segment of a patient's electrogram is the typically flat segment of an electrogram that occurs just before the R wave. For the purposes of this specification, the terms “detection” and “identification” of a cardiac event have the same meaning. A beat is defined as a sub-segment of an electrogram segment containing exactly one R wave.

Heart signal parameters are defined to be any measured or calculated value created during the processing of one or more beats of electrogram data. Heart signal parameters include PQ segment average value, ST segment average value, R wave peak value, ST deviation, ST shift, average signal strength, T wave peak height, T wave average value, T wave deviation, heart rate and R-R interval.

### SUMMARY OF THE INVENTION

The present invention is an emergency room triage system (ERTS) designed to facilitate rapid diagnosis of cardiac events including ST segment related cardiac events from patients with implanted cardiac devices.

The ERTS features of the present invention are applicable to cardiosavers, pacemakers and ICDs or any other implantable device having the capability to detect cardiac events. The cardiosaver is described by Fischell et al. in U.S. Pat. Nos. 6,112,116, 6,272,379 and 6,609,023 which are incorporated herein by reference. The ERTS is designed to display (and/or print) recorded electrogram data and other information downloaded from the implantable device to shorten the time from patient arrival to treatment.

Specifically, the present invention triage system includes a graphical user interface (GUI) designed to display real time and recorded electrogram data that have been downloaded from an implanted device. The recorded data include the following:
1. recent electrogram data recorded in the previous time period (e.g. 24 hours), and
2. event-related electrogram data stored following the detection by the implant of a cardiac event. Event-related electrogram data include the electrogram data whose analysis resulted in the detection and baseline electrogram data used for comparison by the detection algorithms in the implant.
3. trend statistical data such as histogram data that can be used to track ST segment levels over prolonged periods of time.

It is also envisioned that the cardiosaver, pacemaker, ICD and/or pacemaker/ICD combination device would have sensors for recoding of other physiological data including blood pressure, oxygen levels, blood sugar levels and temperature. Associated with such sensors, the ERTS would include the capability to display these additional data to facilitate diagnosis of the patient's condition.

Additionally, the ERTS might include external sensing instruments in the emergency room such as 12-lead electrocardiogram systems, blood pressure sensors and temperature sensors. In this way, the ERTS would begin to resemble the technology envisioned by the original STAR TREK series created by Gene Roddenberry where the sick bay diagnostic beds would display a wide range of physiological data for a recumbent patient.

It is envisioned that external sensing instruments and/or implant access transceiver 20 could be built into a diagnostic bed whereby contact with the patient and patient's implant 5 is made automatically when the patient lies down on the bed. It is also envisioned that the external sensing instruments could be embedded into patient clothes such as the hospital gown. Furthermore, it is envisioned that communication between the ERTS 30 and these external sensing instruments could be via a direct cable or a wireless connection using technologies such as Bluetooth, RF telemetry, and 802.11a-g.

The preferred embodiment of the present invention ERTS would be a touch-screen computer with an implant access transceiver that provides the RF communications link to the implant allowing implant data to be downloaded to and displayed by the touch-screen computer. The implant access transceiver may be built in or attached to the touch-screen computer. A preferred embodiment would have the implant access transceiver attached to the touch-screen computer with a connecting cable. The implant access transceiver would use long range and/or short range data communication. Purely short range data communication would be designed to work with pacemakers and ICDs having only short range telemetry where the implant access transceiver would be placed over the implant site.

Better still would be the use of long range telemetry as described by Fischell et al. in the above referenced patents. However, it may be more efficient to utilize a combination of short and long range data communication to increase the battery life of the implant. The combination of short and long range communication is the preferred embodiment of the present invention. For example, an emergency room might have the ERTS system attached to the wall next to a bed or chair or on a movable cart. An arriving patient would be put in the bed or chair, and the treating medical practitioner would place the implant access transceiver relatively close (typically within 6 inches) to the patient's implant and use the near field telemetry receiver of the implant to initiate long range data communication. The implant access transceiver could then be replaced in its location near the touch-screen computer (e.g. a cradle or a Velcro attachment). The download of data to the ERTS would then begin. Once the data are downloaded, the medical practitioner would use the GUI of the touch-screen computer (or digitizer stylus), to select the data to be displayed and could initiate printing of either the entire data set or the portion being displayed. Thus, another (optional) component of the ERTS would be a printer attached or wirelessly connected to the touch-screen computer using a standard protocol such as Bluetooth or 802.11 a, b or g.

Finally, it is always a challenge to emergency room medical practitioners to access a medical history for an incoming patient in an emergency situation. The capacity to store a patient's relevant medical history data within the implant memory and to display that history using the ERTS would also significantly reduce the time to treatment. Such medical history data could include current medications, allergies, medical insurance information, family history, prior cardiac events, etc.

As ERTS becomes widely used, it is envisioned that large numbers of patients without cardiac implants might receive a very small body-powered implant, such as those used for tracking endangered species, that would provide only the medical history data. In either case, being able to quickly display and print the patient's medical history data would also reduce the time to treatment as compared with having the patient or a family member fill out the appropriate forms.

An additional aspect of the present invention is a miniature data implant having the patient's medical history that works in conjunction with the ERTS. The data implant may be powered from the outside during data communication with the ERTS or by a power source within the patient's body including batteries, miniature fuel cells, kinetic power sources (e.g. as in a self winding watch), thermal power sources or solar power sources. It is envisioned that the miniature data implant might also contain the temperature and pressure sensors mentioned above.

Thus it is an object of this invention to have an emergency room triage system designed to automatically download and display electrogram data captured by an implanted medical device following establishment of data communication between the emergency room triage system and the implant.

Another object of this invention is to have an emergency room triage system with a touch-screen display or digitizer stylus/pen used to select the subset of electrogram data to be displayed.

Still another object of the present invention is to have an emergency room triage system having an attached implant access transceiver having only short range telemetry, both short and long range telemetry and only long range telemetry.

Still another object of the present invention is to have an emergency room triage system with an attached printer.

Yet another object of the present invention is to have an emergency room triage system that can display both recent electrogram data and cardiac-event-related electrogram data.

Yet another object of the present invention is to have an emergency room triage system that can display medical history downloaded from an implanted medical device.

Yet another object of the present invention is to have an emergency room triage system that can display histogram data downloaded from an implanted medical device.

Yet another object of the present invention is to have an emergency room triage system that will sense and display additional physiological data including, but not limited to, temperature, blood pressure, oxygen levels and blood sugar levels.

These and other objects and advantages of this invention will become obvious to a person of ordinary skill in this art upon reading of the detailed description of this invention including the associated drawings as presented herein.

### BRIEF DESCRIPTION OF THE DRAWINGS

FIG. 1 illustrates a Guardian system for the detection of a cardiac event and for warning the patient that a cardiac event is occurring.

FIG. 2 is a block diagram of a cardiosaver system.

FIG. 3 shows the components of the ERTS with the ERTS display showing the patient's medical history data.

FIG. 4 is an example of the ERTS display of alarm related electrogram data downloaded from a cardiosaver.

FIG. 5 is an example of the ERTS display of recent electrogram data downloaded from a cardiosaver.

FIG. 6 is an example of the ERTS real time physiological data display.

### DETAILED DESCRIPTION OF THE INVENTION

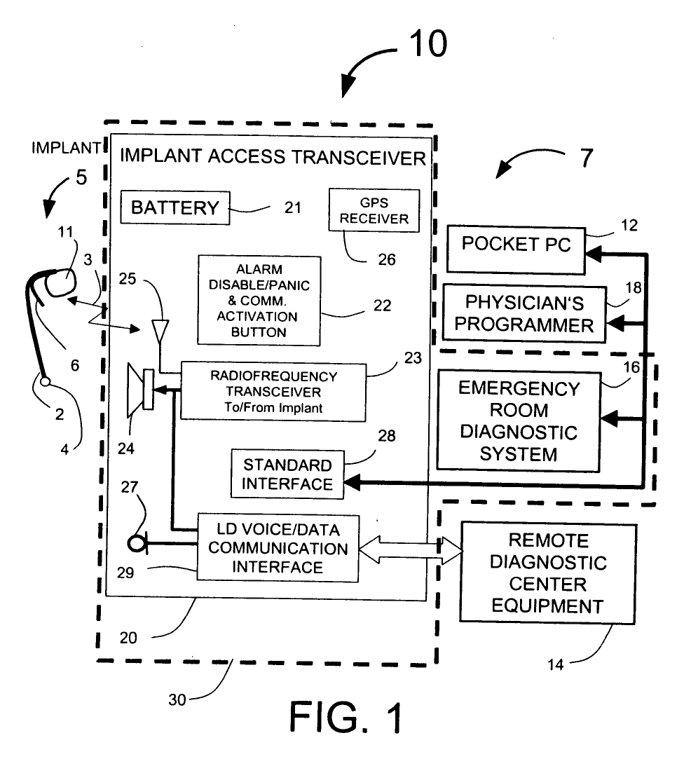

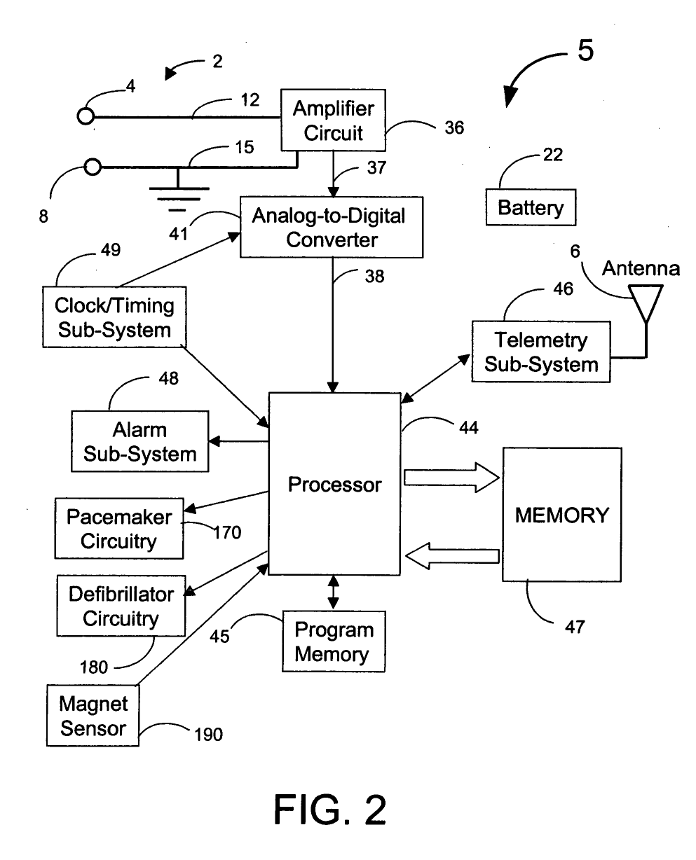

FIG. 1 illustrates one embodiment of the Guardian system 10 consisting of an implanted cardiosaver system 5 and external equipment 7. The cardiosaver system 5 includes a cardiosaver 11, an antenna 6 and an electrode 4 that is part of a lead 2. The cardiosaver 11 includes electronic circuitry that can detect a cardiac event such as an acute myocardial infarction or arrhythmia and can warn the patient with an internal alarm signal when the event occurs. The cardiosaver 11 can store the patient's electrogram for later readout and can send and receive wireless signals 3 to and from the external equipment 7 via the antennas 6 and 25. The functioning of the cardiosaver system 5 will be explained in greater detail with the assistance of FIG. 2.

The cardiosaver system 5 has at least one lead 2 with at least one electrode 4. In fact, the cardiosaver system 5 could utilize as few as one lead or as many as three, and each lead could have as few as one electrode or as many as eight electrodes. The lead 2 in FIG. 1 would advantageously be placed through the patient's vascular system with the electrode 4 being placed into the apex of the right ventricle. For example, the lead 2 could be placed in the right ventricle or right atrium or the superior vena cava similar to the placement of leads for pacemakers and ICDs. The metal case of the cardiosaver 11 could serve as an indifferent electrode with the electrode 4 being the active electrode. Alternately, the lead 2 in FIG. 1 could be placed through the patient's vascular system with the electrode 4 being placed into the apex of the left ventricle.

The lead 2 could advantageously be placed subcutaneously at any location where the electrode 4 would provide a good electrogram signal indicative of the electrical activity of the heart. Again for the lead 2, the case of the cardiosaver 11 of the cardiosaver system 5 could be an indifferent electrode and the electrode 4 would be the active electrode. Although the Guardian system 10 described herein can readily operate with only two electrodes or with one electrode and the case of the cardiosaver being the other electrode, it is envisioned that multiple electrodes used in monopolar or bipolar configurations could be used.

FIG. 1 also shows the external equipment 7 that consists of a physician's programmer 18, a pocket PC 12, the equipment 14 in a remote diagnostic center and the emergency room triage system 30 which includes an implant access transceiver 20 and the emergency room diagnostic system 16. The external equipment 7 provides the means to interact with the cardiosaver system 5. These interactions include programming the cardiosaver 11, retrieving data collected by the cardiosaver system 5 and handling alarms generated by the cardiosaver 11. It should be understood that the cardiosaver system 5 could operate with some but not all of the external equipment 7.

The implant access transceiver 20 includes a battery 21, an alarm disable/panic & communications activation button 22, a radio frequency transceiver 23, a speaker 24 an antenna 25, and a standard interface 28 for providing wired or wireless communication with the pocket PC 12, emergency room diagnostic system 16, or physician's programmer 18. The implant access transceiver 20 may also include an optional microphone 27 and GPS satellite receiver 26. A long distance voice/data communications interface 29 provides connectivity to the remote diagnostic center equipment 14 through voice and data telecommunications networks. For example, the microphone 27 and speaker 24 could be used for wired or wireless telephone calls to and from a medical practitioner at a remote diagnostic center. A built-in modem as part of the interface 29 would allow data to be transmitted to and from the remote diagnostic center equipment 14 over a voice connection. Alternately, a data communications capability of the interface 29 could allow data to be sent or received through a wired or wireless data network. The implant access transceiver 20 may be a separate unit that can be carried by the patient and used by the patient's physician as the data interface to the cardiosaver system 5 or it may also be built into the pocket PC 12, physician's programmer 18 or emergency room diagnostic system 16.

The physician's programmer 18 shown in FIG. 1 is to used to set and/or change the patient medical history data and operating parameters of the implanted cardiosaver system 5 and to read out data stored in the memory of the cardiosaver 11 such as stored electrogram segments as described by Fischell et al. in U.S. Pat. No. 6,609,023.

The pocket PC also described by Fischell et al. in U.S. Pat. No. 6,609,023 can provide the patient or physician the ability to check the status of the cardiosaver 11 and display a limited set of electrogram data downloaded from the cardiosaver 11.

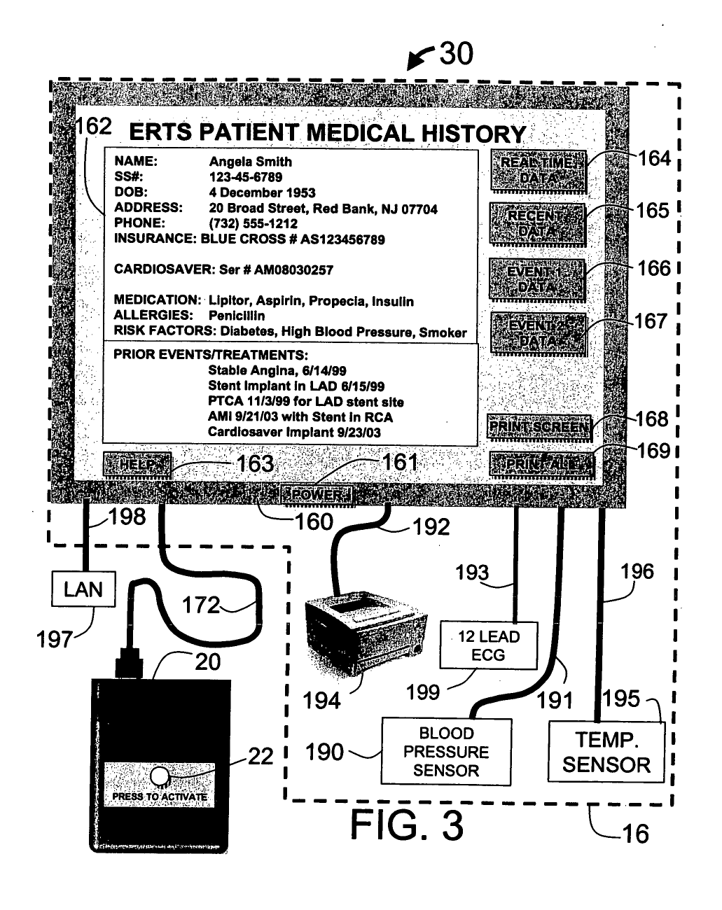

The emergency room diagnostic system 16 is a more sophisticated system than the pocket PC as it can download, display and print all of the data stored within the cardiosaver 11 and would, in its preferred embodiment, use a touch screen display to facilitate triage of patients arriving in an emergency room who have the cardiosaver system 5. This should greatly reduce the time from arrival at the emergency room until treatment for cardiosaver system patients having a cardiac event. The combination of the implant access transceiver 20 and the emergency room diagnostic system 16 form the Emergency Room Triage System (ERTS) 30. The ERTS 30 is designed to reduce the time required for a patient arriving at an emergency medical facility to be rapidly processed and treated as compared to current methods that include the filling out of forms relating to medical history and insurance, finding a portable ECG machine, placing surface leads onto the patient, collecting 12-lead ECG data, and then reading the output of the 12-lead trace. For a patient with an implanted cardiosaver, the present invention includes a method of triage that includes the following steps:
1. Have the patient sit or lie within 6 feet of the implant access transceiver 20 of the ERTS 30.
2. Activate the long range telemetry between the cardiosaver 5 and the implant access transceiver 20 (this would take a few seconds).
3. Quickly download, from the cardiosaver to the ERTS, the stored patient medical history data that then can be displayed and/or printed in the hospital's preferred format.
4. While the medical history is being displayed (and/or printed), the electrogram data stored within the cardiosaver is transmitted to the emergency room diagnostic system 16, the data are then displayed by the ERTS 30 and/or printed on an attached printer 194 (see FIG. 3).
5. Have a medical practitioner review the ST segment levels of the electrogram data displayed by the ERTS 30 or the print out from the printer 194 to confirm or deny the presence of an ST-segment-related cardiac event.
6. If an ST-segment-related cardiac event is diagnosed, rapidly provide the best available treatment.

An implant access transceiver 20 might also be carried by the patient. If a cardiac event is detected by the cardiosaver system 5, an internal alarm signal (typically a vibration or electrical tickle) is generated by the cardiosaver system 5 and an alarm message is sent by a wireless signal 3 to the patient's alarm transceiver 20 via the antennas 6 and 25. When the alarm is received by the alarm transceiver 20, an external alarm signal (typically a sequence of sounds) is generated by the external alarm transceiver 20 and played through the loudspeaker 24 to warn the patient that a cardiac event has occurred. Examples of such sounds include a periodic buzzing, a sequence of tones and/or a speech message that instructs the patient as to what actions should be taken. Furthermore, the alarm transceiver 20 can, depending upon the nature of the signal 3, send an outgoing message to the remote diagnostic center equipment 14 to alert medical practitioners that a cardiosaver system alarm has occurred. The medical practitioners can then utilize the voice communications capabilities of the remote diagnostic center equipment 14 to call back the patient similar to the call that occurs to drivers through the ONSTAR service when their car's air bags deploy in an accident. The optional GPS receiver 26 would allow the data sent to the remote diagnostic center equipment 14 to include patient location to facilitate the summoning of emergency medical services.

The preferred embodiment of the present invention includes long range data communications between the cardiosaver and ERTS 30 such that the communication will work at a distance separation between the antennas 6 and 25 of greater than 1 foot. This is compared with current pacemaker and ICD telemetry systems requiring the data access operate at separations of much less than one foot.

The button 22 will turn off both the internal alarm signal of the implant 5 and the external alarm signal sound being emitted from the loudspeaker 24. An additional feature of the patient's transceiver 20 (i.e. one not connected into an ERTS or programmer), is that if no alarm is occurring, then pressing the alarm button 22 will place a voice and/or data call to the remote diagnostic center similar to the call that is placed when the ONSTAR button is pressed in a car equipped to access the ONSTAR service. Patient location information from the GPS receiver 26 and a subset of patient medical history and electrogram data may be sent as well to the medical practitioners at the remote diagnostic center. The remotely located medical practitioner could then analyze the electrogram data and call the patient back to offer advice as to whether this is an emergency situation or the situation could be routinely handled by the patient's personal physician at some later time.

The implant access transceiver 20 that is part of the ERTS could be the same design as the one carried by the patient; however, they might have different internal programming.

FIG. 2 is a block diagram of the cardiosaver system 5. The electrode 4 connects with wire 12 to the amplifier circuit 36 that is also connected by the wire 15 to the case 8 acting as an indifferent electrode. The lead 2 includes the electrode 4 together with the wire 12 for bringing an electrogram signal into the amplifier circuit 36. The amplified electrogram signals 37 from the amplifier circuit 36 are converted to digital signals 38 by the analog-to-digital converter 41. The digital electrogram signals 38 are then sent to the processor 44. The processor 44 in conjunction with the memory 47 can process the digital signals 38 according to the programming instructions stored in the program memory 45. This programming (i.e. software) enables the cardiosaver system 5 to detect the occurrence of an ST-segment-related cardiac event. An ST-segment-related cardiac event is a cardiac event that is indicated by a change in the shape or level of the ST segment and includes ST segment elevation that is typically indicative of an acute myocardial infarction or ST segment depression that is typically indicative of myocardial ischemia.

A clock/timing sub-system 49 provides the means for timing specific activities of the cardiosaver system 5 including the absolute or relative time stamping of detected cardiac events. The clock/timing sub-system 49 can also facilitate power savings by causing components of the cardiosaver system 5 to go into a low power stand-by mode in between times for electrogram signal collection and processing. Such cycled power savings techniques are often used in implantable pacemakers and defibrillators. In an alternative embodiment, the clock/timing sub-system can be provided by a program subroutine run by the central processing unit 44. It is also envisioned that the processor 44 may include an integral or external First-In-First-Out (FIFO) buffer memory to allow saving of data from before the detection of a cardiac event. Techniques for detecting cardiac events by the processor 44 are described by Fischell et al. in U.S. Pat. No. 6,609,023.

An important aspect of the present invention is the filtering of the electrical signals sensed by the electrodes 4 and 8. The preferred embodiment of the present invention cardiosaver 11 will include high pass and/or low pass filtering of the electrical signals in the amplifier circuit 36. An alternative embodiment would introduce filtering in any or all of the following locations:
1. a separate analog filter between the amplifier circuit 36 and analog-to-digital converter 41,
2. a separate digital filter circuit placed between the analog-to-digital converter 41 and the processor 44,
3. digital filtering performed by the processor 44 on the digital signals 38.

The memory 47 includes specific memory locations for patient data, electrogram segment, histogram/statistical data, and other relevant data storage.

It is envisioned that the cardiosaver system 5 could also contain pacemaker circuitry 170 and/or defibrillator circuitry 180 similar to the cardiosaver system described by Fischell et al. in U.S. Pat. No. 6,240,049.

The alarm sub-system 48 contains the circuitry and transducers to produce the internal alarm signals for the cardiosaver 11. The internal alarm signal can be a mechanical vibration, a sound or a subcutaneous electrical tickle or shock.

The telemetry sub-system 46 with antenna 6 provides the cardiosaver 11 with the means for two-way wireless communication to and from the external equipment 7 of FIG. 1. It is also envisioned that short range telemetry such as that typically used in pacemakers and defibrillators could also be applied to the cardiosaver system 5. It is also envisioned that standard wireless protocols such as Bluetooth and 802.11a or 802.11b might be used to allow communication with a wider group of peripheral devices.

A magnet sensor 190 may be incorporated into the cardiosaver system 5. The primary purpose for a magnet sensor 190 is to keep the cardiosaver system 5 in an off condition until it is checked out just before it is implanted into a patient. This can prevent depletion of the battery life in the period between the times that the cardiosaver system 5 is packaged at the factory until the day it is implanted.

FIG. 3 shows an example of components that can be included in the ERTS 30. These components are the implant access transceiver 20 with activation button 22 and connection 172 and the emergency room diagnostic system 16. The emergency room diagnostic system 16 includes the ERTS display 160 showing the patient's medical history data 162 and may also include a printer 194 with connection 192, a blood pressure sensor 190 with connection 191, a 12 lead electrocardiogram system 199 with connection 193, and a temperature sensor 195 with connection 196. The ERTS 30 also may have a connection 198 to the hospital local area network 197 for sharing data from the ERTS 30 with other hospital systems. The connections 172, 191, 192, 193, 196 and 198 may be either physical wired cables or wireless data connections using infra-red or radiofrequency (RF) data transmission. If a wireless connection is used it would preferably use a standardized protocol such as IRDA for infra-red transmission and Bluetooth or 802.11 a, b, or g for RF transmission. The 12-lead electrocardiogram system 199 would typically have a standard PC interface such as the QRS card system from Pulse Biomedical that can connect into a USB or RS-232 serial port.

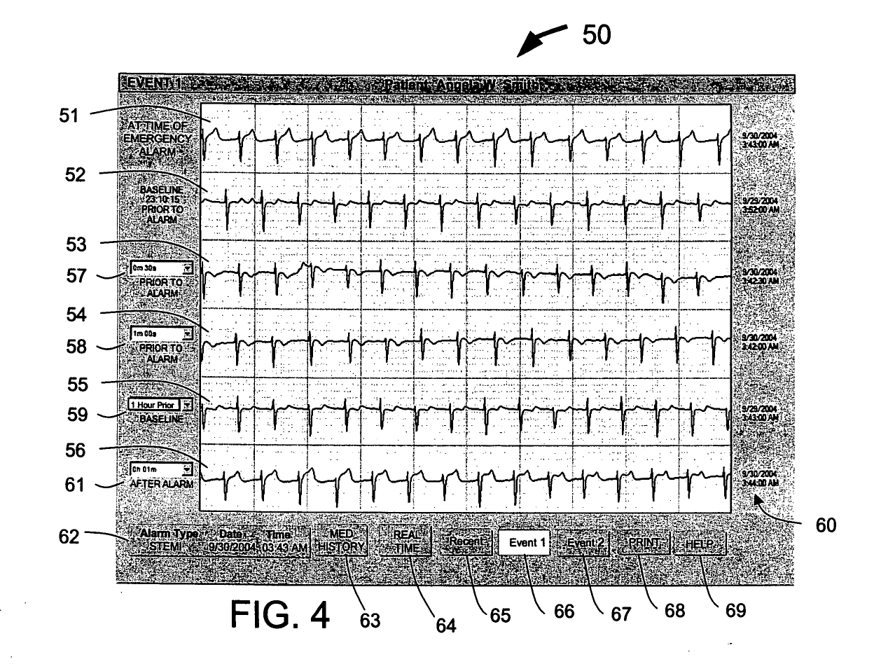

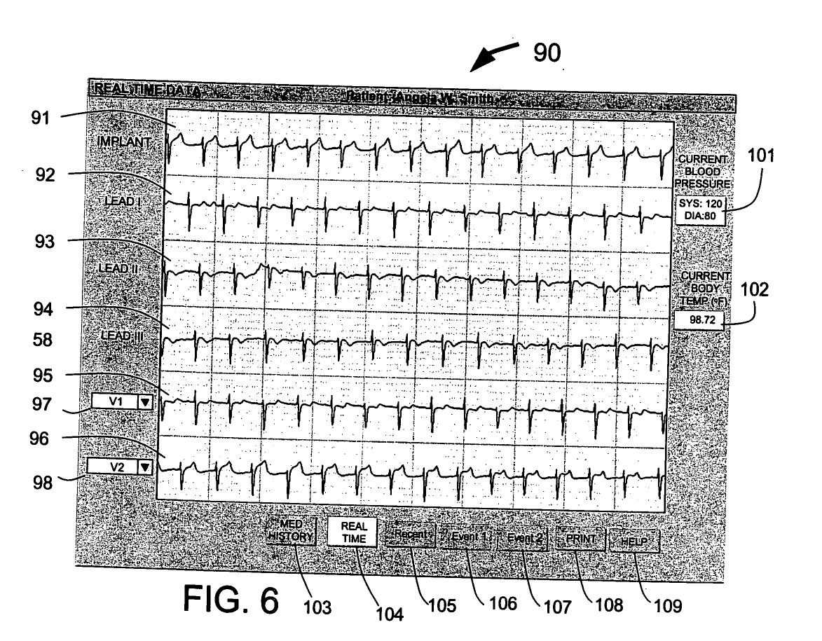

The blood pressure and temperature sensors 190 and 195 allow display of real time patient physical data on the display 160 as display boxes 101 and 102 respectively. It is envisioned that this could be combined with real time display of electrogram data as seen in FIG. 6. The example of the ERTS patient medical history data 162 as shown in FIG. 3 includes the patient name, social security number, date of birth, address, phone, insurance, current medications, allergies, risk factors and a history of prior events and treatments. The ERTS 160 also includes a power button 161 to turn on and off the ERTS 30 and soft control buttons 164 through 167 to switch to the displays of real time data 164, recent data 165, or event data 166 and 167. Soft control buttons are virtual buttons on the display that use the touch-screen or digitizer pen interface. FIGS. 4, 5 and 6 show examples of ERTS displays activated by these buttons. The soft control buttons 168 and 169 are print buttons including a print screen button 168 that will print the data on the current screen, and a print all button 169 that will provide a full print out of all the patient data available in the ERTS 30. When the ERTS 30 is turned on, it would typically show a start screen instructing the medical practitioner to place the implant access transceiver 20 near to the implant 5 of FIG. 1 and depress the button 22. This will initiate a data communications session with the implant 5 and initiate the transmission of data stored in the implant memory 47 of FIG. 2 to the ERTS 30. Once the transmission (that may take a few minutes) is complete, the ERTS display 160 would show the patient medical history 162 and the other control buttons seen in FIG. 3.

Although FIG. 3 shows only two event data buttons 166 and 167, it is envisioned that the ERTS 30 would typically show one button for each cardiac event whose data have been transmitted from the implant 5 to the ERTS 30 during the data communication session. The soft control button 163 provides built in instructions for use of the ERTS 30 and the functions of the display 160. The display 160 would typically be a touch sensitive screen that can be used interacted with by use of a finger or stylus. An attached stylus might be best.

The preferred embodiment of the present invention envisions a Graphical User Interface (GUI) that includes the use of selection boxes with pop up menus (e.g., a windows start button) and soft control buttons (e.g. a windows X button that closes a window), well known in PC software. Such selection boxes and soft control buttons are typically selected using a touch-screen interface as in a PDA or tablet PC or a pointing device like a mouse, touchpad or trackball. However, because of the limited number of buttons needed for the ERTS 30, it is envisioned that actual physical buttons could be utilized by the ERTS 30 instead of soft control buttons shown in FIGS. 3, 4, 5 and 6.

FIG. 4 is an example of the ERTS display 50 initiated by the selection of soft control button 166 of FIG. 3. The display 50 shows the first cardiac event alarm-related electrogram data downloaded from the cardiosaver 5 of FIG. 1. The display 50 shows 6 electrogram segments 51 through 56 related to the emergency alarm that occurred at T=03:43 am as indicated by the segment time indications 60. The electrogram segment 51 is the electrogram segment whose analysis by the cardiosaver system 5 triggered the detection of the cardiac event associated with an emergency alarm. The alarm information box 62 indicates that this detection was of the type STEMI (ST Elevation Myocardial Infarction) and also includes the date and time of the detection. Although the FIG. 4 shows times with that have AM (or PM), a 24 hour time could also be used.

The electrogram segment 52 is the baseline electrogram segment from approximately 24 hours before the time of the alarm. As described by Fischell et al. in U.S. Pat. No. 6,240,049, The T minus 24 hour baseline electrogram segment is utilized by the cardiosaver system 5 ST shift detection algorithm for comparison with current electrogram data. The display 50 also includes the segments 53 through 56 that provide information on the patient's heart both before and after the cardiac event. The segments 53 and 54 are selectable to display any of the electrogram segments from the period just preceding the cardiac event. In this example 53 has been selected to display the T minus 0 minutes 30 seconds electrogram segment and 54 has been selected to display the T minus 1 minute 0 seconds. The selection boxes 57 and 58 typically accessed by the touch-screen interface allow the user to select other recorded electrogram segments from the time period just before the cardiac event. For example, the cardiosaver system 5 might record electrogram segments for 10 seconds every 30 seconds and always have in memory the last 8 electrogram segments. When a cardiac event is detected, these would be saved for later review as the segments 51, 53 and 54. Similarly, if for example the cardiosaver system 5 stores a baseline electrogram segment once per hour, then at the time of a detected cardiac event, these baseline segments would be saved for later review as the electrogram segment 52 (the T-24 hour baseline) and the other baseline segments 55 selectable by the box 59. The cardiosaver system 5 also has the capability to record electrogram data for some period of time after the detection of a cardiac event. These post event electrogram data are shown as the electrogram segment 56 selectable by the selection box 61. The display 50 would typically be a touch-sensitive screen that can be used interacted with by use of a finger or stylus. An attached stylus might be best.

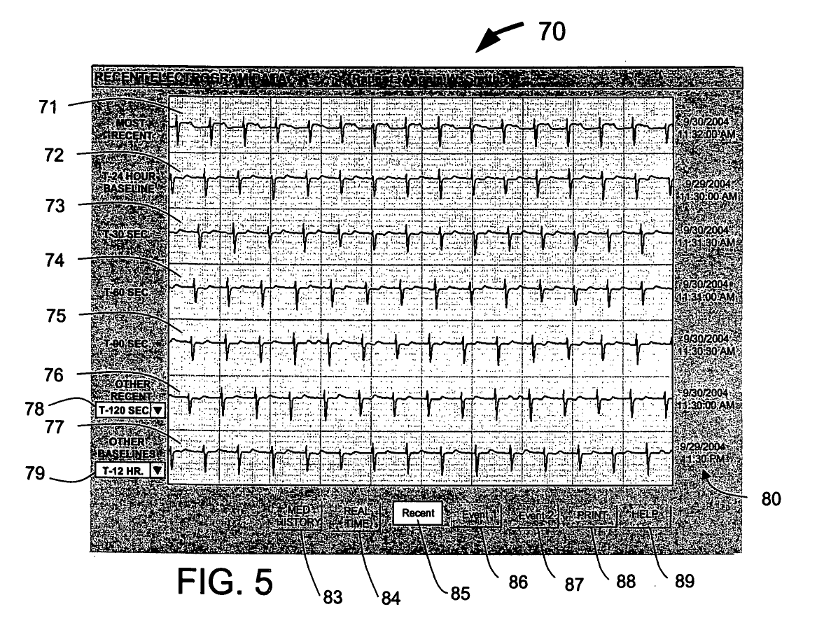

The soft control buttons 63 through 69 provide access to the other functions and screens from the display 50. Button 66 is highlighted on screen 50 to show that this is the display of Event 1. Button 63 will return to the patient medical history screen 160 of FIG. 3. Button 64 will access the real time electrogram display 90 shown in FIG. 6. Button 65 will provide access to the screen 70 of FIG. 5. Button 67 would provide access to the display of electrogram data for Event 2 downloaded from the cardiosaver 5 of FIG. 1. If more than 2 events have been downloaded from the cardiosaver system 5 of FIG. 1 then it is envisioned that there could be additional event display buttons (e.g. EVENT 3, EVENT 4 etc) or the event 2 button 67 could instead be labeled “OTHER EVENTS”) that would enable an additional menu used to select the other event to be displayed. Button 68 will access print controls allowing the printing of either the data of the display 50 or all of the downloaded data also printed from the soft control button 169 of FIG. 3. The soft control button 69 provides built in instructions for use of the ERTS 30 and the functions of the display 50.

FIG. 5 is an example of the ERTS display 70 of recent electrogram data downloaded from the cardiosaver 5 of FIG. 1. The display 70 accessed by the soft control button 165 of FIG. 3 or button 65 of FIG. 4 shows the most recently collected electrogram data downloaded from the cardiosaver 5 of FIG. 1. The display 70 shows 7 electrogram segments 71 through 77. The electrogram segment 71 is the last electrogram segment stored by the cardiosaver 5 just before the download process began. The actual date and time for each electrogram segment 71 through 77 are shown in the corresponding location in the data field 80.

The electrogram segment 72 is the baseline electrogram segment from approximately 24 hours before the collection of the electrogram segment 71. The segments 73, 74 and 75 show the other electrogram segments from the two minutes just preceding the download. In this case they show the T minus 30 seconds, T minus 60 seconds and T minus 90 seconds, where T is the time of collection for the most recent electrogram segment 71. The selection boxes 78 and 79 allow the user to select other recorded electrogram segments from the time period before the download. For example, the selection box 78 could select fairly recent electrogram data (e.g. T minus 120 seconds) and would typically have a pop up menu with available choices. The selection box 79 could be used to select the display of other hourly baseline electrogram data recordings (e.g. T minus 12 hours).

The display 70 would typically be a touch sensitive screen that can be used interacted with by use of a finger or stylus. An attached stylus might be best.

The soft control buttons 83 through 89 provide access to the other functions and screens from the display 70. Button 85 is highlighted on screen 70 to show that this is the display of recently collected electrogram data. Button 83 will return to the patient medical history screen 160 of FIG. 3. Button 84 will provide access to the real time electrogram data display screen 90 shown in FIG. 6. Button 86 will provide access to the screen 50 of FIG. 4. Button 87 would provide access to the display of electrogram data for Event 2 downloaded from the cardiosaver 5 of FIG. 1. If more than 2 events have been downloaded from the cardiosaver system 5 of FIG. 1 then it is envisioned that there could be additional event display buttons (e.g. EVENT 3, EVENT 4 etc) or the event 2 button 87 could instead be labeled “OTHER EVENTS”) that would enable an additional menu used to select the other event to be displayed. Button 88 will access print controls allowing the printing of either the data of the display 70 or all of the downloaded data also printed from the soft control button 169 of FIG. 3. The soft control button 89 provides built in instructions for use of the ERTS 30 and the functions of the display 70.

FIG. 6 is an example of the ERTS display 90 of real time electrogram data transmitted by the implant 5 of FIG. 1 and electrocardiogram data received from the 12-lead system 199 of FIG. 3. The display 90 is accessed by the soft control button 164 of FIG. 3, button 64 of FIG. 4 or button 84 of FIG. 5. The display 90 shows real time heart signal data both from the implant 5 of FIG. 1 and/or the 12-lead system 199 of FIG.3. The display 90 shows 6 electrogram/electrocardiogram signals 91 through 96. The electrogram segment 91 is the real time display of electrogram data transmitted from the cardiosaver 5.

The electrocardiogram signals 92 through 96 come from the 12-lead system 199. In this example, the signals 92, 93 and 94 are the standard 12-lead displays of LEADS I, II and III respectively. The signals 95 and 96 chosen by selection boxes 97 and 98 are other 12-lead signal displays (e.g. V1, V2 etc.).

The display 90 would typically be a touch-sensitive screen that can be interacted with by use of a finger or stylus. An attached stylus might be best.

The soft control buttons 103 through 109 provide access to the other functions and screens from the display 90. Button 104 is highlighted on screen 90 to show that this is the display of real time data. Button 103 will return to the patient medical history screen 160 of FIG. 3. Button 105 will provide access to the recent electrogram data display screen 70 shown in FIG. 5. Button 106 will provide access to the screen 50 of FIG. 4. Button 107 would provide access to the display of electrogram data for Event 2 downloaded from the cardiosaver 5 of FIG. 1. Button 108 will access print controls allowing the printing of either the data of the display 90 or all of the downloaded data also printed from the soft control button 169 of FIG. 3. The soft control button 109 provides built in instructions for use of the ERTS 30 and the functions of the display 90. It is also envisioned that all of the processing techniques described herein for the implantable cardiosaver 5 of FIG. 1 are applicable to a guardian system configuration using skin surface electrodes and a non-implanted cardiosaver. If a non-implanted cardiosaver using skin surface electrodes is used then the term electrogram would be replaced by the term electrocardiogram.

Various other modifications, adaptations, and alternative designs are of course possible in light of the above teachings. Therefore, it should be understood at this time that, within the scope of the appended claims, the invention can be practiced otherwise than as specifically described herein.

1. An emergency room triage system for patient diagnosis, the system including:
an implanted cardiac device recording electrogram data associated with the detection of a cardiac event;
communication means for receiving electrogram data from the implanted cardiac device; and
a visual display for displaying said electrogram data recorded by the implanted cardiac device, the visual display including means for displaying electrogram data associated with an ST segment related cardiac event.

2. The system of claim 1, wherein said implanted cardiac device includes a cardiosaver.

3. The system of claim 1, wherein said implanted cardiac device includes a pacemaker.

4. The system of claim 1, wherein said implanted cardiac device includes a cardiac defibrillator.

5. The system of claim 1, where the ST segment related cardiac event is an acute myocardial infarction.

6. The system of claim 1, where the ST segment related cardiac event is an ST elevation.

7. The system of claim 1, where the ST segment related cardiac event is an ST depression.

8. The system of claim 1, where the ST segment related cardiac event is exercise induced ischemia.

9. The system of claim 1, where the electrogram data displayed includes baseline electrogram data used by the implanted cardiac device to detect the ST segment related cardiac event.

10. The system of claim 1, where the electrogram data displayed includes electrogram data recorded during the day before the detection of the coronary event.

11. The system of claim 1, where the electrogram data displayed includes electrogram data recorded during the day following the detection of the coronary event.

12. The system of claim 1, where the visual display is a touch screen display.

13. The system of claim 1, where the visual display displays patient related medical history data.

14. The system of claim 1, further including a printer.

15. The system of claim 1, further including means for displaying electrogram data transmitted in real time from the implanted cardiac device.

16. The system of claim 1, further comprising means for displaying heart signal data from a 12 lead electrocardiogram system.

17. The emergency room triage system of claim 1, further including a blood pressure sensor.

18. The emergency room triage system of claim 1, further including a temperature sensor.

19. The system of claim 1, where the implanted cardiac device and said emergency room triage system each includes antennae to facilitate wireless communication therebetween, the wireless communication being designed to operate at separation of greater than one foot between the implanted cardiac device's antenna and emergency room triage system's antenna.

20. The system of claim 1, further including a separate implant access transceiver providing the communication between the implanted cardiac device and the emergency room triage system.

21. The system of claim 1, where the visual display is provided by a tablet PC running a version of the Microsoft Windows operating system.

22. The system of claim 1, further including means for displaying arrhythmia related cardiac events.

23. The system of claim 22, where the arrhythmia is selected from the group consisting of tachycardia, bradycardia, unsteady heart rate, bigeminal rhythm, premature ventricular contractions, premature atrial contractions and atrial fibrillation.

24. The system of claim 1, where the implanted cardiac device includes at least one electrode located within the heart.

25. The system of claim 24, where the electrode located within the heart is located within the right ventricle.

26. The system of claim 24, where the electrode located within the heart is located within the right atrium.

27. The system of claim 24, where the implanted cardiac device includes at least one electrode located subcutaneously.

28. The system of claim 27, where at least one subcutaneously located electrode is located on the patient's left side.

29. The system of claim 1, where the emergency room triage system is mounted on a wall of the emergency room.

30. The system of claim 1, where the emergency room triage system is used in conjunction with a moveable cart.

31. The system of claim 1, further including a local area network connection to other computers.

32. The system of claim 1, where the implanted cardiac device includes at least one temperature sensor.

33. The system of claim 1, where the implanted cardiac device includes at least one pressure sensor.

34. The system of claim 4, where the implanted cardiac defibrillator further includes a pacemaker circuitry.

35-66. (canceled)
